(19) United States
(12) Patent Application Publication
(10) Pub. No.: US 2026/0034379 A1
(43) Pub. Date: Feb. 5, 2026

(54) Transcranial Magnetic Stimulation Probe

(71) Applicant: United States Government as represented by the Department Of Veterans Affairs, Washington, DC (US)

(72) Inventors: Jauhtai Cheng, Palo Alto, CA (US); Jerome Yesavage, Palo Alto, CA (US); Margaret Windy McNerney, Palo Alto, CA (US); Alesha Heath, Palo Alto, CA (US); Daid Piekarski, Palo Alto, CA (US)

(21) Appl. No.: 19/285,269

(22) Filed: Jul. 30, 2025

Related U.S. Application Data
(60) Provisional application No. 63/677,117, filed on Jul. 30, 2024.

(51) Int. Cl. A61N 2/00; A61N 2/02; G01R 13/02

(52) U.S. Cl. CPC A61N 2/006; A61N 2/02; G01R 13/0272

(57) ABSTRACT

A probe for use with a transcranial magnetic stimulation (TMS) device can include a coil having a first end and a second end. A first lead wire electrically coupled to the first end of the coil, and a second lead wire electrically coupled to the second end portion of the coil. The coil has a central axis and a major dimension from 2 mm to 8 mm measured perpendicularly to the central axis. The coil can have from about five to about twenty turns.

### CROSS-REFERENCE TO RELATED APPLICATION

This application claims priority to and the benefit of the filing date of U.S. Provisional Patent Application No. 63/677,117, filed Jul. 30, 2024, the entirety of which is incorporated by reference herein for all purposes.

### FIELD

This disclosure relates to transcranial magnetic stimulation (TMS) probes that can be used with small animals.

### BACKGROUND

Transcranial magnetic stimulation (TMS) is a technology that applies time-varying magnetic field to treat neurological and psychological conditions. The time-varying magnetic field produced by TMS devices secondarily induces a pulsating electric field according to Faraday's law of electromagnetic induction. The therapeutic effect of TMS is derived from the interaction between the induced electric field and neurons in the brain. Specifically, the therapeutic effect of TMS depends on the strength and temporal and spatial characteristics of the induced electric field. The strength of the induced electric field is proportional to the magnitude of the current that flows through the TMS coil, while the spatial characteristics of the electric field depends on the configuration of the TMS coil.

Researchers are studying how modifying the design of the TMS coil may affect the therapeutic efficacy of TMS. A sensor/probe capable of providing quantitative information on the spatial characteristics of the induced field is essential for the development of better TMS device. To protect human subjects, before a new TMS device can be used in humans, animal models are often employed to obtain preliminary data to justify human studies. Existing electric field probes are ill-suited for measuring the electric field generated by TMS coils for animal studies.

### SUMMARY

Transcranial magnetic stimulation (TMS) uses pulsating electric field to improve brain function. Experimental TMS devices are used to treat animal subjects and collect animal data. This treatment of animal subjects can permit better design of TMS devices for humans without exposing research subjects to unnecessary risks. A disclosed probe is provided that is suitable to measure the electric field generated by the animal TMS device. The disclosed probe is physically small, yet has a high power of amplification to detect weak electric field.

The probe can include a coil having a first end and a second end. A first lead wire electrically coupled to the first end of the coil, and a second lead wire electrically coupled to the second end portion of the coil. The coil has a central axis and a major dimension from 2 mm to 8 mm measured perpendicularly to the central axis. The coil can have from about five to about twenty turns. The coil can comprise wire having an American Wire Gauge (AWG) size of 20 or smaller.

Systems and methods of using the probe are disclosed.

In one aspect, a method comprises the step of obtaining a first signal from a probe corresponding to current induced in the probe. The probe comprises a coil having a first end portion and a second end portion, a first lead wire electrically coupled to the first end portion of the coil, and a second lead wire electrically coupled to the second end portion of the coil. The first signal corresponds to an electromagnetic field emitted from a TMS coil configured for use with a small animal.

These and other objects and features of the present invention will become more fully apparent from the following description and appended claims, or may be learned by the practice of the invention as set forth hereinafter.

### BRIEF DESCRIPTION OF THE DRAWINGS

FIG. 1 is a schematic diagram of an exemplary probe and system in accordance with embodiments disclosed herein. The length of wire of each turn is π·D. Accordingly, the wire can have a total length of n·π·D, where n is the number of turns.

FIG. 2 is an image of a coil of an exemplary probe, shown against an imperial scale for size reference. As shown, the coil has a length of about ¼ inch, or about 5 mm.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, use of “substantially” (e.g., “substantially parallel”) or “generally” (e.g., “generally planar”) should be understood to include embodiments in which angles are within ten degrees, or within five degrees, or within one degree.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

### Motivation

Transcranial magnetic stimulation (TMS) uses pulsating electric field to improve brain function. Experimental TMS devices are used to treat animal subjects and collect animal data. This treatment of animal subjects can permit better design of TMS devices for humans without exposing research subjects to unnecessary risks. It is difficult to characterize the electric field of animal TMS device using the conventional electric field probe because: 1) animal TMS device is two orders of magnitude smaller than the human TMS device; 2) the conventional electric field probe has a physical dimension about an order of magnitude larger than the animal TMS device; and 3) the animal TMS device generates electric field 3 orders of magnitudes smaller than that of the human TMS device. To overcome such difficulty, the disclosed probe is provided that is suitable to measure the electric field generated by animal TMS device. The disclosed probe is physically small, yet has a high power of amplification to detect weak electric field.

### Introduction

TMS machines generate extremely low frequency electromagnetic field (ELF). To measure the strength of ELF electric field, Anthony Valentino described a probe of 6.3 cm in diameter capable of measuring electric field of extremely low frequencies. It has sensitivity of 10 mV/m. Additional details of the probe of Valentino are disclosed in Valentino, A. R. (1972 July). A small ELF electric field probe. In 1972 IEEE International Electromagnetic Compatibility Symposium Record (pp. 1-6). IEEE, which is incorporated by reference herein in its entirety for all purposes. The probe described by Valentino is physically too large to characterize the electric field generated by small coils for animal studies, which have the diameter of about 3 mm-4 mm.

In practice, the diameter of a TMS coil for human subjects is about 10 cm. Conventional electric field probes have an inner diameter of 20 mm and outer diameter of 26 mm, with an average circumference of about 70 mm. The electric field generated by a TMS coil for human applications is about 100-200 V/m at its peak. Conventional electric field probes have spatial resolution and sensitivity adequate to quantify the spatial characteristics of the electric field generated by the human TMS coils. However, the experimental TMS coil for animal study are much smaller, about 3 mm-4 mm in diameter. The electric field generated by the animal TMS coil is weak, about 100 mV/m at its peak. The electric field declines sharply with distance, to a level of less than 10 mV/m at a distance of about 10 mm from the coil. The dimension and sensitivity of the electric field probes for human coil are not suitable for studying the electric field induced by the animal TMS coil. The disclosed probe overcomes this difficulty.

### Probe

Disclosed herein, in various aspects, is a probe configured to measure the weak electric field of a pulsating magnetic field by applying the Faraday's law of electromagnetic induction. The probe uses a coil of conducting wire to amplify the induced electric field, thus allowing for measurement of the weak field generated by the animal TMS coil as further disclosed herein.

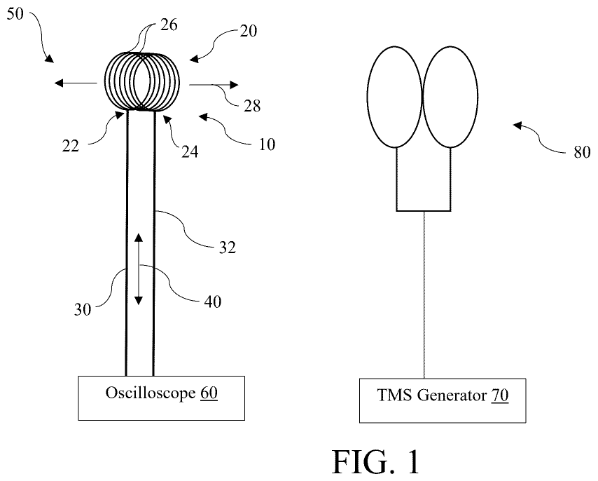

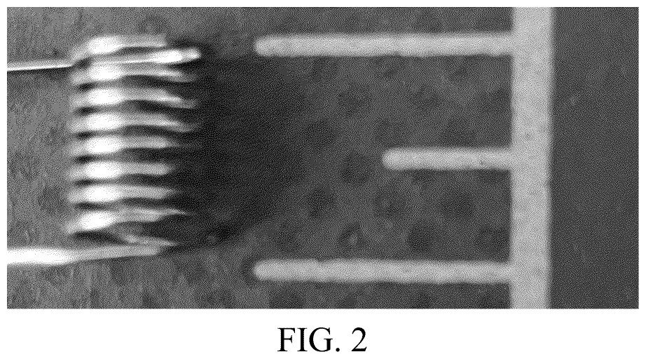

FIGS. 1-2 illustrate the physical construct of a probe 10 in accordance with embodiments disclosed herein. The probe 10 can comprise a coil 20 including a first end portion having a first end 22, a second end portion having a second end 24, and a plurality of turns 26 (e.g., loops) positioned between the first and second ends 22, 24. The probe 10 can further comprise a first lead wire 30 and a second lead wire 32. The first lead wire 30 can couple to the first end 22 of the coil 20, and the second lead wire 32 can couple to the second end 24 of the coil. The first and second ends 22, 24 of the coil 20 can be physically close to each other but not in direct contact. In this way, a differential in electric field due to difference in spatial location between the first and second ends 22, 24 can be minimized. For example, in some aspects, the first and second ends 22, 24 can be within 12 mm, or within 11 mm, or within 10 mm, or within 9 mm, or within 8 mm, or within 7 mm, or within 6 mm, or within 5 mm, or within 4 mm, or within 3 mm, or within 2 mm of one another.

In some aspects, the coil 20 can comprise or consist of copper. In further or alternative aspects, the coil can comprise or consist of other metallic wire, such as, for example, Chromel, a nickel-chromium alloy. Optionally, in these aspects, the coil 20 can comprise or consist of enameled copper wire. In some aspects, the coil 20 can comprise, or be formed from, a wire having a size from American Wire Gauge (AWG) 20 to AWG 36. In this way, the coil 20 can be compact and permit the first and second ends 22, 24 to be positioned close to each other.

In some aspects, the first and second lead wires 30, 32 can have a greater size (e.g., thickness) than that of the coil 20. For example, one or both of the first and second lead wires 30, 32 can have a size that is AWG 20 or thicker. For example, the first and second lead wires 30, 32 can each have a size that is from about AWG 12 to about AWG 20, or from about AWG 12 to about AWG 16. In this way, the first and second lead wires 30, 32 can provide physical integrity for the probe 10. In some optional aspects, the first and second lead wires 30, 32 can comprise or consist of copper. More generally, the first and second lead wires can comprise metallic wire.

In various optional aspects, the coil 20 can have from about five turns 26 to about twenty turns, or from seven turns to fifteen turns. For example, in some aspects, the coil 20 can have from about eight turns 26 to about fifteen turns, or about ten turns. In some aspects, the coil 20 can have five, six, seven, eight, nine, ten, eleven, twelve, thirteen, fourteen, fifteen, sixteen, seventeen, eighteen, nineteen, or twenty turns 26.

In some aspects, the coil 20 can have circular turns. That is, the coil 20 can have a consistent radial dimension from a central axis 28 of the coil. In other aspects, the coil can be oblong. For example, the coil can appear oval such that when viewing the coil 20 along the central axis 28, the coil can have an oval profile. In additional aspects, the turns 26 of the coil 20 can have square, rectangular, triangular, pentagonal, hexagonal, octagonal, or any suitable profile.

The coil 20 can have a major dimension, defined as a maximum dimension perpendicular to the central axis 28. For example, the major dimension can be a diameter. In some aspects, the major dimension (e.g., diameter) of the coil 20 can be from about 2 mm to about 8 mm, or from about 2 mm to about 5 mm, or about 3.5 mm, or about 4 mm.

A system 50 can comprise a probe 10 and an oscilloscope 60 coupled thereto. For example, the first and second lead wires 30, 32 can be electrically coupled to the oscilloscope 60. The first and second lead wires 30, 32 can conduct electricity between the coil 20 and the oscilloscope 60. When a TMS device is turned on, the induced electric field can be picked up and amplified by the coil assembly. The amplification power is based on the fact that the induced electric field is a non-conservative field. Thus, the electric field can be integrated over an entire span of the coil 20. For example, by providing a plurality of turns, signal can be amplified. For example, within the application of small animal TMS coils, a single coil can provide a signal on the order of less than 1 mV, which can be on the order of noise. By providing a plurality of turns, the signal can be amplified by the number of turns. The electric field can be registered at the oscilloscope as ΔV, corresponding to a voltage difference between the first and second ends 22, 24 of the coil 20.

Referring to FIG. 2, in some aspects, a coil can comprise 28 AWG wire. In additional aspects, the wire can comprise or consist of Chromel (e.g., 90% nickel, 10% chromium). The coil can have a length along the central axis from about 3 mm to about 8 mm, or about 5 mm. The coil can have a diameter from about 3 mm to about 5 mm, or about 4 mm.

Advantages of the disclosed configuration include:
1. The proximity of the two ends ensures the differential in electric field due to difference in spatial location is minimized.
2. The small size of the coil allows the assumption of relatively uniform electric field strength, E.
3. The length of wire with each turn is easily calculated as π*D, and the total length of the coil is N*π*D.
4. The electric field strength (E) can be calculated by the following equation: E=ΔV/(N*π*D), where ΔV is the voltage difference between the two ends of the coil.
5. Using coil of multiple turns allows for shrinking the size of the probe without compromising the sensitivity.

Due to its small size (for example, about 3.5 mm in diameter), the spatial resolution of the new probe can be an order of magnitude better than that of the currently technology. Due to the high power of amplification, the sensitivity of the probe 10 can be better than that of the conventional probe.

A method of using the probe 10 and system 50 can include positioning the probe in an electromagnetic field and obtaining a first signal from the probe corresponding to a portion of the electromagnetic field passing through the coil 20. For example, the electromagnetic field can be generated by a TMS generator 70 and an associated TMS coil 80. In some aspects, the TMS coil 80 can be configured for use with small animals. For example, the TMS coil 80 can be sized for positioning on a rat or a mouse. Accordingly, in some optional aspects, the TMS coil 80 can have a major dimension that is less than 2 cm, or less than 1.5 cm, or less than 1 cm. The probe 10 can be moved to a second position relative to the TMS coil 80 (either by moving the probe relative to the TMS coil or the TMS coil relative to the probe, or both). A second signal can be obtained from the probe 10. In some aspects, the second position can comprise a translation (i.e., moving the probe 10 along a vertical and/or horizontal axis). In this way, electromagnetic field can be determined for different positions. In other aspects, the second position can comprise a rotation of the coil about an axis 40 transverse to the central axis 28. In this way, a direction of magnetic field can be determined. A plurality of additional signals can be obtained from respective additional positions (rotational and/or translational) of the probe 10 relative to the TMS coil 80. Accordingly, by obtaining a plurality of signals corresponding to different translational and/or rotational positions of the probe 10 relative to the TMS coil 80, an electromagnetic field of the TMS coil can be mapped.

As further explained herein, the disclosed probe can take advantage of the concept of integration. The disclosed probe can provide the fine spatial details of the electric field the probe is measuring. However, a single loop probe of smaller dimension would only be able to integrate the electric field of a short length (that is π·D), with the magnitude of the voltage thus integrated in the order of sub-millivolts for a field generated by an animal coil. This voltage is near the ambient noise level, making such a single-loop probe unsuitable for use. Using the disclosed design of a coil of n number of turns, the electric field is integrated over n·π·D, making the signal n-times stronger than a single-loop probe without the need for using external signal amplifiers. Thus, the disclosed probe acts as a “passive device” that is capable of amplifying signals.

Although the foregoing invention has been described in some detail by way of illustration and example for purposes of clarity of understanding, certain changes and modifications may be practiced within the scope of the appended claims.

What is claimed is:

1. A probe for use with a transcranial magnetic stimulation (TMS) device, the probe comprising:
a coil having a first end portion and a second end portion, wherein the coil has a central axis and a major dimension from 2 mm to 8 mm measured perpendicularly to the central axis, wherein the coil has from five turns to twenty turns;
a first lead wire electrically coupled to the first end portion of the coil; and
a second lead wire electrically coupled to the second end portion of the coil.

2. The probe of claim 1, wherein the coil comprises wire having a size from American Wire Gauge (AWG) 20 to AWG 36.

3. The probe of claim 1, wherein the first and second lead wires are AWG 20 or thicker.

4. The probe of claim 1, wherein the coil has a diameter from 2 mm to 5 mm.

5. The probe of claim 1, wherein the coil has a diameter of about 3.5 mm.

6. The probe of claim 1, wherein the coil has from 7 to 15 turns.

7. The probe of claim 1, wherein the coil has 10 turns.

8. The probe of claim 1, wherein the first and second lead wires comprise copper.

9. The probe of claim 1, wherein the coil comprises a wire having a gauge, wherein the first and second lead wires each have a respective gauge that is larger than the gauge of the wire of the coil.

10. A system comprising:
an oscilloscope; and
a probe as in claim 1, wherein the oscilloscope is in electrical communication with each of the first and second lead wires.

11. The system of claim 10, further comprising a TMS coil.

12. The system of claim 11, wherein the TMS coil is configured for use with a small animal.

13. A method comprising:
obtaining a first signal from a probe corresponding to current induced in the probe, wherein the probe comprises:
a coil having a first end portion and a second end portion;
a first lead wire electrically coupled to the first end portion of the coil; and
a second lead wire electrically coupled to the second end portion of the coil, wherein the first signal corresponds to an electromagnetic field emitted from a TMS coil configured for use with a small animal.

14. The method of claim 13, wherein obtaining the first signal comprises obtaining a reading from an oscilloscope, wherein the oscilloscope is electrically coupled to the first and second lead wires.

15. The method of claim 13, wherein the electromagnetic field generated by the TMS coil induces the current in the probe.

16. The method of claim 15, further comprising changing a relative position between the TMS coil and the probe.

17. The method of claim 16, wherein changing the relative position between the TMS coil and the probe comprises rotating the probe relative to the TMS coil.

18. The method of claim 16, wherein changing the relative position between the TMS coil and the probe comprises translating the probe relative to the TMS coil.

19. The method of claim 13, wherein the coil has a central axis and a major dimension from 2 mm to 8 mm measured perpendicularly to the central axis, wherein the coil has from five turns to twenty turns.

20. The method of claim 13, wherein the coil comprises a wire having a gauge, wherein the first and second lead wires each have a respective gauge that is larger than the gauge of the wire of the coil.
